(19) United States
(12) Patent Application Publication
(10) Pub. No.: US 2024/0352505 A1
(43) Pub. Date: Oct. 24, 2024

(54) METHODS FOR SELECTING ANTIMICROBIALS AGAINST PHYTOPATHOGENS

(71) Applicant: ORGANICIN SCIENTIFIC, INC., Wilmington, DE (US)

(72) Inventors: Griffin O'Driscoll, Northborough, MA (US); Mathew Mitchell, Philadelphia, PA (US); Margaret Riley, Amherst, MA (US)

(21) Appl. No.: 18/292,603

(22) PCT Filed: Jul. 27, 2022
(86) PCT No.: PCT/US2022/038476

§ 371 Date: Jan. 26, 2024

Related U.S. Application Data
(60) Provisional application No. 63/203,622, filed on Jul. 27, 2021.

(51) Int. Cl. C12Q 1/20

(52) U.S. Cl. CPC C12Q 1/20

(57) ABSTRACT

This invention relates to methods of selecting plant-associated bacterial isolates for promoting plant health.

### CROSS REFERENCE

This application claims the benefit of priority of U.S. Provisional Application No. 63/203,622, filed Jul. 27, 2021, the contents of which are hereby incorporated by reference in their entirety for all purposes.

### BACKGROUND

Plant pathogens, or phytopathogens, are a global threat to the world's food supply. Phytopathogens cause numerous diseases such as fire blight of apples, black rot, and bacterial speck of tomato, which result in significant crop losses. The global economic impact of plant pathogens exceeds $1 billion in losses annually.

As the global human population size continues to increase, the ability to feed this population has not kept pace. Efforts to meet this critical need have resulted in a gradual transition to more sustainable agricultural practices. However, crop loss due to infectious disease from phytopathogens creates a considerable challenge as it requires the use of unsustainable management strategies such as the frequent application of antibiotics and other bactericides to crops.

One of the tools available for phytopathogen management is the use of antibiotics, primarily streptomycin and oxytetracycline. Resistance to these antibiotics has been detected in numerous plant pathogens, most notably in Erwinia amylovora, the causal agent of fire blight. Furthermore, while the levels of antibiotics employed in plant agriculture remain limited, its use may be linked to increased levels of antibiotic resistance in human pathogens through the horizontal transfer of resistance genes. The use of broad-spectrum bactericides and antibiotics to limit or eliminate bacterial infections is becoming less effective as levels of resistance increase. Broad-spectrum bactericides and antibiotics also impact not only the target phytopathogen, but also numerous beneficial bacteria, such as plant growth-promoting rhizobacteria (PGPR) found in the plant and soil microbiomes. Accordingly, the use of broad-spectrum bactericides and antibiotics is less desirable from an ecological perspective.

Thus, there is a pressing need to identify and select novel antimicrobials that are effective against one or more phytopathogens for use as alternatives and/or supplements to antibiotics.

### SUMMARY

Disclosed herein, in certain embodiments, are methods of selecting one or more organism-associated bacterial isolates for promoting organism health, including: (a) co-culturing each of the bacterial isolates with an organism-associated pathogen; (b) determining the level of inhibition activity for each bacterial isolate against the organism-associated pathogen; (c) co-culturing each of the bacterial isolates with an organism health-promoting bacteria (e.g., a probiotic); (d) determining the level of inhibition activity for each bacterial isolate against the organism health-promoting bacteria; and (e) selecting the one or more bacterial isolates for promoting organism health if: the bacterial isolate has inhibition activity against the organism-associated pathogen according to step (b); and the bacterial isolate has substantially no inhibition activity against the organism health-promoting bacteria according to step (d). Such a method may, for example, enable the ability to more efficiently and accurately select one or more organism-associated bacterial isolates for the production of bacteriocins and other antimicrobials with the characteristics required to inhibit or kill pathogenic bacteria while having minimal impact on the microbiome of the host, such that commensal and growth-enhancing) bacteria (e.g., probiotic) remain intact.

In some embodiments, the method may further include one or more steps selected from the list including: a) selecting a target organism and reviewing bacteria associated with the organism; b) determining which organism-associated bacteria contribute to the overall health and/or disease-resistance of the organism (e.g., bacteria that are a net benefit to the survival of the host); c) determining which organism-associated bacteria are pathogenic or detrimental to the overall health of the organism (e.g., bacteria that are a net detriment to the survival of the host); d) determining which organism-associated bacteria are dependent on the host organism for survival or reproduction; and/or e) co-culturing each of the bacterial isolates selected from step d) with each of the bacterial isolates selected from step b) and step c).

In some embodiments, the organism is a non-microbial eukaryote.

In some embodiments, the non-microbial eukaryote is an aquatic organism (e.g., fish, shrimp, and oyster), a terrestrial organism (e.g., an animal e.g., plant, cow, chicken, pig, and horse), a human, or a plant.

In some embodiments, the organism is a plant.

In some embodiments, the organism-associated pathogen is a phytopathogen.

In some embodiments, health-promoting includes growth-promoting and/or producing a bacteriocin or antimicrobial.

In some embodiments, the organism health-promoting bacteria (e.g., probiotic) is a rhizobacteria, a probiotic, or a plant commensal.

Disclosed herein, in certain embodiments, are methods of selecting one or more plant-associated bacterial isolates for promoting plant health, including: (a) co-culturing each of the bacterial isolates with a phytopathogen; (b) determining the level of inhibition activity for each bacterial isolate against the phytopathogen; (c) co-culturing each of the bacterial isolates with a plant growth-promoting rhizobacteria, a probiotic, or a plant commensal; (d) determining the level of inhibition activity for each bacterial isolate against the plant growth-promoting rhizobacteria, probiotic, or plant commensal; and (e) selecting the one or more bacterial isolates for promoting plant health if: the bacterial isolate has inhibition activity against the phytopathogen according to step (b); and the bacterial isolate has substantially no inhibition activity against the plant growth-promoting rhizobacteria, probiotic, or plant commensal according to step (d).

Disclosed herein, in certain embodiments, are methods of selecting one or more plant-associated bacterial isolates for promoting plant health, including: (a) co-culturing each of the bacterial isolates with at least 5 different phytopathogens; (b) determining the level of inhibition activity for each bacterial isolate against each phytopathogen; (c) co-culturing each of the bacterial isolates with at least 5 different plant growth-promoting rhizobacteria, probiotics, and/or plant commensals; (d) determining the level of inhibition activity for each bacterial isolate against each plant growth-promoting rhizobacteria, probiotic, or plant commensal; (e) selecting the one or more bacterial isolates for promoting plant health if: the one or more bacterial isolates has inhibition activity against each of the phytopathogens according to step (b); and the one or more bacterial isolates has substantially no inhibition activity against each the plant growth-promoting rhizobacteria, probiotic, or plant commensal according to step (d).

In some embodiments, promoting plant health includes promoting one or more plant health parameters selected from the following list: (i) disease resistance; (ii) the ability to produce or change the concentration of indoleacetic acid, gibberellic acid, cytokinins and/or ethylene; (iii) the ability to perform asymbiotic N2 fixation; (iv) the ability to produce siderophores, antibiotics, or cyanide; (v) the ability to solubilize mineral phosphates and other nutrients; and (vi) the ability to change performance of symbiotic N2 fixation, nodulation, or nodule occupancy.

In some embodiments, the phytopathogen includes Erwinia amylovora, Agrobacterium tumefaciens, Pseudomonas syringae pv. tomato or other P. syringae pathovars, P. cichorii, Xanthomonas campestris pv. armoraciae and pv. vesicatoria, Ralstonia solanacearum, X. axonopodis pv. glycines, X. axonopodis pv. vasculorum, X. vasicola pv. holcicola, X. vasicola pv. vasculorum, X. campestris pv. campestris or other pathogenic Xanthamonas, or Xylella fastidiosa.

In some embodiments, the plant growth-promoting rhizobacteria includes Alcaligens species, Arthrobacter species, Bacillus cereus, B. circulans, B. coagulans, B. licheniformis, B. megaterium, B. pseudomycoides, B. pumilus, and other Bacillus species, Enterobacter aerogenes, E. cloacae, E. taylorae, Klebsiella oxytoca, K. pneumoniae, P. aeruginosa and other Pseudomonas species, Paraburkholderia species, Serratia marcescens, S. plymuthica, S. rubidaea and other Serratia species, and Acinetobacter baumannii or other Acinetobacter species.

In some embodiments, the plant commensal includes Arabidopsis species, Citrobacter amalonaticus, C. freundii, C. koseri or other Citrobacter species, Escherichia coli, Hafnia alvei or other Hafnia species, Morganella morganii or other Morganella species, Pantoea vagans or other Pantoea species, Proteus mirabilis, Providencia rettgeri, Salmonella enterica or other Salmonella species, or Stenotrophomonas maltophili.

In some embodiments, substantially no inhibition comprises a zone of inhibition of less than about 5 mm (e.g., less than about 4 mm, less than about 3 mm, less than about 2 mm, or less than about 1 mm). Alternatively, for example, substantially no inhibition includes inhibiting less than 33% (e.g., less than 32%, 31%, 30%, 25%, 20%, 15%, 10%, 5%, 1%, or 0.5%) of organism health-promoting bacteria (e.g., PGPR) isolates tested. In some embodiments, inhibition includes a zone of inhibition of greater than about 5 mm (e.g., greater than about 6 mm, greater than about 7 mm, greater than about 8 mm, greater than about 9 mm, or greater than about 10 mm).

In some embodiments, co-culturing includes co-culturing on a transwell plate, in a microfluidic platform, on a solid support, or in a broth. For example, in some embodiments. culturing includes co-culturing on a solid support. In some embodiments, a solid support includes a plate, a three-dimensional scaffold, a hydrogel, or a microarray. In some embodiments, the plate is a nutrient agar plate. Alternatively, for example, in some embodiments, co-culturing includes co-culturing in a broth.

Disclosed herein, in certain embodiments, are methods of the invention further including determining a ratio of inhibitory activity for each of the one or more bacterial isolates and selecting the one or more bacterial isolates for promoting plant health if the ratio is greater than 1.

Disclosed herein, in certain embodiments, are A method of determining whether one or more plant-associated bacterial isolates is likely to have inhibition activity against a phytopathogen, including: a) obtaining a control bacterial isolate having inhibition activity against the phytopathogen; b) determining the phylogenetic distance for each of the one or more plant-associated bacterial isolates against the control bacterial isolate; an c) determining the one or more plant-associated bacterial isolates as likely to have inhibition activity against the phytopathogen if the phylogenetic distance to the control bacterial isolate according to step (b) is less than or equal to 0.040.

The present disclosure provides the insight that mutualistic bacteria—bacteria that have a reason to produce bacteriocins—are the best bacterial candidate when screening for antimicrobial substances, such as bacteriocins. In accordance with this principle, the methods disclosed herein have the significant advantage of finding novel bacteriocins more efficiently than random high-throughput screening. Further, the bacteriocins that are discovered using the disclosed methods are more likely to have high-specificity, as they would have no evolutionary advantage to inhibit bacteria that doesn't kill their host and would have an evolutionary disadvantage to inhibit bacteria that makes their host healthy. By utilizing evolutionary game theory and classic natural selection, the screening methods disclosed herein take advantage of the different strategies that bacteria use to compete against one another for resources needed to survive and reproduce.

### BRIEF DESCRIPTION OF THE DRAWINGS

FIG. 1 shows a screening assay of plant-associated bacteria isolates for producing narrow-spectrum bacteriocins and other antimicrobials that may promote plant health. The X-axis “sensitive” lawns of bacteria (i.e., these bacteria were tested to see if they could be inhibited by some antimicrobial substance produced by another bacteria), while the Y-axis shows whether the tested bacteria were a “producer” (i.e., bacteria were spotted on a lawn to see if they would inhibit other bacteria). Each black dot is an instance of a single isolate of bacteria from the Y-axis inhibiting a single isolate of bacteria from the X-axis. Abbreviations: PP, phytopathogen; PGPR, plant growth-promoting rhizobacteria; PC, plant commensal.

FIG. 2 shows a phylogenetic representation of inhibitory interactions. The genera containing plant pathogens are identified by * and the PGPR genera are identified by +. A heat map of inhibitory activity was then superimposed onto this phylogeny. The genera along the top of the figure (X-axis) are the potentially inhibitory taxa. Their ability to inhibit members of each genus is plotted against the tips of the phylogenetic tree (Y-axis). The degree of inhibition is indicated in the key and ranges from no inhibition (clear box) to 100% inhibition (darkest grey).

FIG. 3 is a plot of weighted average degree of sensitivity for certain genera.

FIG. 4 is a plot of the ratios of inhibition for the 44 species that compares the relative number of PP or PGPR isolates inhibited by members of a species. The ratio for each species is indicated to the right of the species name, with a black dot indicating the average ratio for that species, and the horizontal lines indicating the breadth of ratios calculated for isolates within the species.

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

As used in the description of the invention and the appended claims, the singular forms “a,” “an” and “the” are intended to include the plural forms as well, unless the context clearly indicates otherwise.

The terms “substantially” or “about” are used interchangeably herein and mean an acceptable error for a particular value as determined by one of ordinary skill in the art, which depends in part on how the value is measured or determined. In certain embodiments, the terms “substantially” or “about” mean within 10%, 9%, 8%, 7%, 6%, 5%, 4%, 3%, 2%, 1%, 0.5%, or 0.05% of a given value or range. For example, in some embodiments, “substantially no inhibition” means 0%+10% inhibition, wherein any suitable unit may be used. In some embodiments, “substantially no inhibition” means a zone of inhibition of less than about 5 mm (e.g., less than about 4 mm, less than about 3 mm, less than about 2 mm, or less than about 1 mm). Alternatively, for example, “substantially no inhibition” includes inhibiting less than 33% (e.g., less than 32%, 31%, 30%, 25%, 20%, 15%, 10%, 5%, 1%, or 0.5%) of organism health-promoting bacteria (e.g., PGPR) isolates tested. The term “about,” as used herein when referring to a measurable value such as an amount of a compound or agent, dose, time, temperature, and the like, is meant to encompass variations of ±10%, ±5%, ±1%, ±0.5%, or even ±0.1% of the specified amount.

As used herein, “and/or” refers to and encompasses any and all possible combinations of one or more of the associated listed items, as well as the lack of combinations when interpreted in the alternative (“or”).

Moreover, any feature or combination of features set forth herein can be excluded or omitted.

Unless otherwise defined, all technical and scientific terms used herein have the same meaning as commonly understood by one of ordinary skill in the art. The terminology used in the description herein is for the purpose of describing particular embodiments only and is not intended to be limiting.

The term “phytopathogen” as used herein refers to a pathogenic organism that infects a plant. Examples of phytopathogens include: Erwinia amylovora (the causative agent of fire blight), Agrobacterium tumefaciens, Pseudomonas syringae pv. tomato (which causes bacterial speck) or other P. syringae pathovars, P. cichorii, Xanthomonas. campestris pv. armoraciae and pv. vesicatoria (which cause bacterial leaf spot), Ralstonia solanacearum (bacterial wilt), X. axonopodis pv. glycines (bacterial pustule), X. axonopodis pv. vasculorum (gumming disease), X. vasicola pv. holcicola (streaky spot), X. vasicola pv. vasculorum (gumming disease), X. campestris pv. campestris (black rot) or other pathogenic Xanthomonas, and Xylella fastidiosa.

The term “plant pathogens” or PP as used herein refer to bacteria known to cause disease in plants

The terms “organism health-promoting bacteria” or “probiotic” are used interchangeably herein to mean live microorganisms that provide health benefits when consumed by an organism, often by improving or restoring the gut flora.

The term “plant growth-promoting rhizobacteria” or PGPR as used herein refer to bacteria known to benefit a plant's growth and/or health. PGPR may benefit a plant's health by suppressing of plant diseases or inducing of disease resistance in plants. PGPR are a heterogeneous group of bacteria that can be found in the rhizosphere, at root surfaces and in association with roots. PGPR are known to be present in the soil and to live in association with plants. PGPR have the ability to colonize the roots and express their plant growth promotion activities in the rhizosphere.

In some embodiments, the PGPR promote growth in plants. In some embodiments, the PGPR promote disease resistance in plants. In some embodiments, the PGPR promote plant health. In some embodiments, the PGPR promote plant yield. Reducing the overall damage of plants and plant parts often results in healthier plants and/or in an increase in plant vigor and yield.

PGPR can promote or improve the extent or quality of plant growth and/or health directly or indirectly. The direct promotion of plant growth and/or health by PGPR include providing the plant with a plant growth-promoting substances that is synthesized by the bacterium, facilitating the uptake of certain plant nutrients from the environment, or improving physiological metabolic processes such as N2 fixation, phosphate and potassium solubilization and phytohormone auxin indole-3-acetic acid (IAA) production. The indirect promotion of plant growth and/or health includes when PGPR lessen or prevent the deleterious effect of one or more phytopathogenic micro-organisms or suppress diseases (e.g., by secreting antimicrobials). The mechanisms by which PGPR promote plant growth and/or health may include (i) the ability to produce or change the concentration of plant growth regulators like indoleacetic acid, gibberellic acid, cytokinins and ethylene, (ii) asymbiotic N2 fixation, (iii) antagonism against phytopathogenic microorganisms by production of siderophores, antibiotics and cyanide, and (iv) solubilization of mineral phosphates and other nutrients. Some PGPR may promote plant growth and/or health indirectly by affecting symbiotic N2 fixation, nodulation, or nodule occupancy.

Examples of PGPR include: Alcaligens species, Arthrobacter species, Bacillus cereus, B. circulans, B. coagulans, B. licheniformis, B. megaterium, B. pseudomycoides, B. pumilus, and other Bacillus species, Enterobacter aerogenes, E. cloacae, E. taylorae, Klebsiella oxytoca, K. pneumoniae, P. aeruginosa and other Pseudomonas species, Paraburkholderia species, Serratia marcescens, S. plymuthica, S. rubidaea and other Serratia species, and Acinetobacter baumannii or other Acinetobacter species.

The term “plant commensals” or PC as used herein refer to plant and soil dwelling bacteria that are not known to either help or harm a plant. Examples of PC include: Arabidopsis species, Citrobacter amalonaticus, C. freundii, C. koseri or other Citrobacter species, Escherichia coli, Hafnia alvei or other Hafnia species, Morganella morganii or other Morganella species, Pantoea vagans or other Pantoea species, Proteus mirabilis, Providencia rettgeri, Salmonella enterica or other Salmonella species, and Stenotrophomonas maltophili. Commensal bacteria may be selected based on their general ubiquity in nature and lack of both beneficial or detrimental impacts on agricultural plants.

The term “rhizosphere” a used herein refers to a narrow zone of soil that surrounds and is influenced by plant roots, gives home to an overwhelming variety of organisms, in particular microorganisms such as bacteria. This complex microbial community has profound effects on plant growth since it facilitates nutrient absorption and provides health protection to plants.

Examples of properties of “promoting plant growth” include increased plant yield, improved seedling vigor, improved root development, improved plant growth, improved plant health, improved appearance, improved resistance to plant pathogens, reduced pathogenic infection, or a combination thereof.

Examples of properties of “promoting disease resistance” include antagonistic properties to confer protection against plant pathogenic infections and/or to treat or control plant pathogenic infections.

The term “plant health” generally includes various improvements of plants. For example, advantageous properties are improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (e.g., reduced production and/or inhibition of reception), tillering increase, increase in plant height, bigger leaf blade, less dead basal leaves, stronger tillers, greener leaf color, pigment content, photosynthetic activity, less input needed (e.g., fertilizers or water), less seeds needed, more productive tillers, earlier flowering, early grain maturity, less plant verse (e.g., lodging), increased shoot growth, enhanced plant vigor, increased plant stand, and early and better germination.

With regard to the methods according to the present invention, improved plant health refers to improved plant characteristics including: crop yield, increased plant yield, more developed root system, improved root growth, improved root size maintenance, improved root effectiveness, tillering increase, increase in plant height, bigger leaf blade, less dead basal leaves, stronger tillers, greener leaf color, photosynthetic activity, more productive tillers, enhanced plant vigor, and increased plant stand.

### Antimicrobials

Bacteria are believed to have coevolved with plants, meaning that their genetic changes are influenced by the plant they live on and vice versa. Alternatively, other bacteria have evolved to be pathogenic, meaning they generally gain resources and reproduce by infecting a plant, typically resulting in death or decreased health of the plant. Third, bacteria have also evolved to be mutualistic, meaning they gain resources by living alongside a plant and in return provide resources to the benefit of the plant.

Since mutualistic bacteria require a host plant to get the resources they need to survive, it is in their best interest to protect and promote the health of their host. Death of the host plant means death of the mutualistic bacteria and, therefore, no reproduction. Since pathogenic bacteria need to infect a plant to survive, it is therefore also in the best interest of the mutualistic bacteria to have or produce means of killing off pathogenic bacteria.

Bacteria produce numerous antimicrobial substances including conventional small molecule antibiotics, ribosomally-synthesized peptides and proteins known as bacteriocins and microcins, bacteriocin-like inhibitory substances, as well as organic acids, hydrogen peroxide, and diacetyl. Many of these inhibitory antimicrobial substances have narrow inhibitory spectra. Some of these inhibitory substances are produced in response to signals received from a potential competitor which then elicits an antagonistic response. Growth inhibition of one isolate by another may be due to the production of a range of antimicrobial substances, including conventional small molecule antibiotics, ribosomally-synthesized peptides and proteins known as bacteriocins and microcins, bacteriocin-like inhibitory substances, as well as organic acids, hydrogen peroxide, and diacetyl.

The present disclosure describes methods of selecting or identifying antimicrobials having inhibitory activity against one or more phytopathogens. The present disclosure also describes methods of selecting or identifying one or more plant-associated bacterial isolates for promoting plant growth and/or health (e.g., promoting disease resistance).

### Bacteriocins

The term “bacteriocin” as used herein refer a class of proteinaceous toxins produced by bacteria to inhibit the growth of similar or closely related bacterial strain(s).

Bacteriocins are an example of inducible antimicrobials. Bacteriocins are a frequently encountered family of bacterial antimicrobials. Bacteriocins are ribosomally synthesized antimicrobial peptides and proteins produced by many species of bacteria, which often preferentially target close relatives of the producing species. Several studies have suggested that virtually all bacteria are able to produce bacteriocins.

Bacteriocins possess characteristics that make them particularly appealing for consideration as an alternative to antibiotics. Bacteriocins exhibit little to no toxicity to humans, with several identified as GRAS (generally recognized as safe) by the FDA and used in food preservation for over fifty years. Because they are highly specific in their breadth of activity, bacteriocins active only against specific plant pathogens have been identified, and thus pose little risk of inhibiting members of the plant microbiome, as well as resulting in reduced selective pressures for evolving resistance. There is a rapidly growing literature on industrial bacteriocin fermentation methods and numerous companies now economically manufacture bacteriocins for a variety of uses, such as preservatives or flavor enhancers.

One method to induce bacteriocin production includes placing isolates in competition for limited resources, such as when they are co-cultured on a nutrient agar plate. In some embodiments, isolates may be co-cultured on a nutrient agar plate or nutrient broth. Co-culturing by nutrient broth may include 96-well screening methods. Another method of inducing bacteriocin production includes eliciting the SOS regulatory pathway through introduction of stress, such as by adding Mitomycin C which damages DNA. Methods to observe the resulting inhibitory activity are well known to those of ordinary skill in the art.

### Methods of Identifying and Selecting

Disclosed herein, in certain embodiments, are methods to identify or select one or more plant-associated bacterial isolates having inhibition activity against one or more phytopathogens. In some embodiments, the one or more bacterial isolates have substantially no inhibitory activity against members of the plant and rhizosphere microbiomes. In some embodiments, the one or more bacterial isolates have substantially no inhibitory activity against bacteria identified as PGPR. In some embodiments, the phenotypic inhibition observed in bacteria are the result of the production of bacteriocins and bacteriocin-like inhibitory substances.

In some embodiments, the bacterial isolates have substantially no activity against beneficial members of the plant and soil microbiomes. In some embodiments, the bacterial isolates have substantially no activity against growth-promoting bacteria.

### EXAMPLES

The disclosure is further illustrated by the following examples. The examples are provided for illustrative purposes only, and are not to be construed as limiting the scope or content of the disclosure in any way.

### Example 1: Co-Culture Inhibition Assays and Phenotypic Analysis

The inhibitory interactions were examined between bacteria commonly found associated with a representative sample of crop plants. Isolates of plant-associated bacteria were investigated for the production of inhibitory substances (e.g., narrow-spectrum bacteriocins and other antimicrobials) as well as sensitivity to these inhibitory substances.

Bacterial isolates were employed in co-culture inhibition assays to determine if the isolates were capable of inhibiting each other's growth. The assay takes advantage of the fact that numerous studies suggest that many of the inhibitory substances produced by bacteria are bacteriocins and that, upon induction or signaling, bacteriocin producing bacteria will rapidly produce large quantities of their bacteriocins.

In the “all by all” phenotypic screen, each isolate was considered to be a potential inhibitory isolate as well as potentially sensitive to inhibition by other isolates. A lawn of each isolate was created on a nutrient agar plate and cultures of each isolate were then spotted (co-cultured) on the lawn. Inhibition was observed as a zone of inhibited growth of the lawn around the spotted, potentially inhibitory isolates. Inhibition was determined subjectively based on a blind consensus after observing the zone of inhibition, and accepted if 2 out of 3 replicates produced the same consensus.

In some embodiments, the level of inhibition activity for a bacterial isolate is by observation of the zone of inhibition. In some embodiments, observation of the zone of inhibition includes measuring the diameter of the zone of inhibition, observing the transparency of the zone of inhibition, or observing the distribution of activity in the zone of inhibition. In some embodiments, a bacterial isolate is determined to have inhibition activity against a phytopathogen if the zone of inhibition is greater than or equal to about 5 mm in diameter. In some embodiments, a bacterial isolate is determined to have inhibition activity against a phytopathogen if the zone of inhibition is substantially not transparent. In some embodiments, a bacterial isolate is determined to have inhibition activity against a phytopathogen if the zone of inhibition has a substantially consistent distribution of activity. In some embodiments, a bacterial isolate is determined to have substantially no inhibition activity against a plant growth-promoting rhizobacteria if the zone of inhibition is less than about 5 mm in diameter. In some embodiments, a bacterial isolate is determined to have substantially no inhibition activity against a phytopathogen if the zone of inhibition is substantially transparent.

Table 1 provides a list of the 217 isolates of plant-associated bacteria included in the study and the functional categories they were assigned.

TABLE 1
Bacterial Isolates

| | | | Category |
|---|---|---|---|
| Category | Species | Isolates | References |
| Phyto- | Erwinia amylovora | 20 | [4] |
| Pathogens | Pseudomonas cichorii | 2 | [5] |
| | Pseudomonas syringae | 12 | [4] |
| | Ralstonia solanacearum | 2 | [4] |
| | Xanthomonas axonopodis pv. glycines | 1 | [14] |
| | Xanthomonas axonopodis pv. vasculorum | 1 | [15] |
| | Xanthomonas campestris pv. armoracide | 1 | [17] |
| | Xanthomonas campestris pv. campestris | 9 | [4] |
| | Xanthomonas campestris pv. vesicatoria | 2 | [16] |
| | Xanthomonas vasicola pv. holcicola | 1 | [15] |
| | Xanthomonas vasicola pv. vasculorum | 1 | [15] |
| Plant | Alcaligenes spp. | 1 | [1] |
| Growth- | Bacillus cereus | 1 | [1][6] |
| Promoting | Bacillus circulans | 1 | [1][6] |
| Rhizobacteria | Bacillus coagulans | 1 | [1][6] |
| | Bacillus licheniformis | 1 | [1][2][6] |
| | Bacillus megaterium | 4 | [1][2][3][6] |
| | Bacillus pseudomycoides | 2 | [1][6] |
| | Bacillus pumilus | 2 | [1][2][6] |
| | Bacillus spp. | 5 | [1][2][6] |
| | Bacillus subtilis | 1 | [1][2][6] |
| | Enterobacter aerogenes | 9 | [1] |
| | Enterobacter cloacae | 10 | [1][3] |
| | Enterobacter taylorae | 10 | [1] |
| | Klebsiella sp. | 3 | [1] |
| | Klebsiella oxytoca | 15 | [1] |
| | Klebsiella pneumoniae | 3 | [1] |
| | Pseudomonas aeruginosa | 12 | [1][6] |
| | Serratia marcescens | 9 | [1][3][6] |
| | Serratia plymuthica | 5 | [1][6] |
| | Serratia rubidaea | 1 | [1][6] |
| Plant | Acinetobacter baumannii | 13 | [1][6] |
| Commensals | Citrobacter amalonaticus | 4 | [7][9] |
| | Citrobacter freundii | 10 | [7][9] |
| | Citrobacter koseri | 2 | [7][9] |
| | Escherichia coli | 3 | [8] |
| | Hafnia alvei | 10 | [18] |
| | Morganella morganii | 2 | [19] |
| | Pantoea vagans | 1 | [9] |
| | Proteus mirabilis | 1 | [10] |
| | Providencia rettgeri | 1 | [11] |
| | Salmonella enterica | 1 | [12] |
| | Stenotrophomonas maltophilia | 21 | [13] |

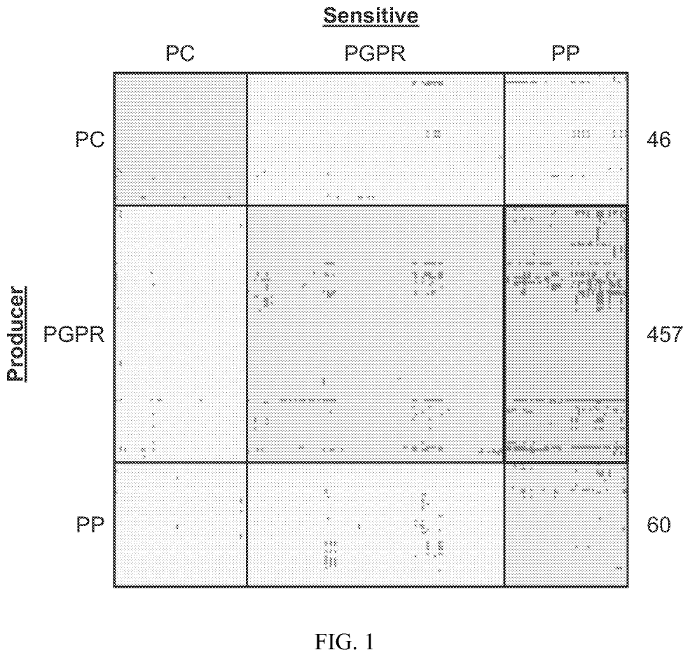

Cultures of the 217 isolates were made by single colony inoculation of 10 mL LB broth and incubation overnight at 30° C. in a shaking incubator. Bacterial lawns were created by aliquoting 100 uL of each culture on an LB plate and spreading with sterile glass beads. Once the lawns were dry, 5 uL of each culture was aliquoted on each lawn. The LB plates were then incubated overnight at 30° C. Each strain was scored for production of an inhibitory substance and/or sensitivity to inhibitory substances. To score for production, a blind consensus was conducted on the zone of inhibition, which was based on the understanding that any presence of inhibition indicates some activity on behalf of the spotted isolate. In general, a zone of inhibition was observed as a circular zone of inhibition no less than 5 mm in diameter, that was transparent (as opposed to hazy, which would indicate some presence of lawned bacteria), and crisp (e.g., lacked spottiness, indicating a consistent distribution of activity). A consensus was reached by two researchers who gave agreeing scores, or by three researchers when one score disagreed. The isolates were tested in at least two assays, and in three assays if the consensus of the first two assays disagreed. An isolate was recorded as a producer after at least two assays were scored with a consensus indicating production. A total of 47,089 independent assays were conducted in triplicate, generating 141,267 data points. FIG. 1 illustrates such an experiment. In particular, the X-axis of FIG. 1 shows “sensitive” lawns of bacteria (i.e., these bacteria were tested to see if they could be inhibited by some antimicrobial substance produced by another bacteria), while the Y-axis of FIG. 1 shows whether the tested bacteria were a “producer” (i.e., bacteria were spotted on a lawn to see if they would inhibit other bacteria). Each black dot is an instance of a single isolate of bacteria from the Y-axis inhibiting a single isolate of bacteria from the X-axis.

Generally, in such an experiment, one can consider plant commensal (PC) bacteria to be a control, as they neither harm nor benefit the plant (e.g., neutral bacteria e.g., live on a plant but do not need the plant), and therefore should have minimal evolutionary pressure to compete against any bacteria. By contrast, plant growth-promoting rhizobacteria (PGPR) are the mutualistic bacteria in this experiment, as they benefit the plant and the plant benefits from them. PGPR also need the plant to survive.

A diverse selection of phytopathogenic species of bacteria were selected and analyzed to determine how sensitive they were to the inhibitory substances produced by other bacteria. Table 2 provides a summary of the inhibitory interactions observed among the 217 bacterial isolates during co-culture. The 217 bacterial isolates from 19 genera and 44 species were investigated, including known PP (24%), growth-promotingPGPR (50%), and PC (26%).

FIG. 1 provides an illustration and Table 2 provides a breakdown of the inhibitory activity and sensitivity within each functional category.

As visualized by FIG. 1, PC neither killed nor died frequently, meaning that they were unlikely to produce a bacteriocin and unlikely to be targeted by a bacteriocin. As visualized by FIG. 1, PGPR bacteria were the most likely to inhibit a PP bacteria, and generally did not inhibit PC or other PGPR bacteria. When comparing the frequency of inhibition (number of times an isolate of bacteria was seen to inhibit other bacteria) for PC against that of PGPR, PC bacteria had a total of 46 instances of inhibition against PP bacteria, whereas PGPR bacteria had a total of 457 instances of inhibition against PP bacteria, supporting the conclusion that bacteria are more likely to survive if they are capable of inhibiting PGPR. In particular, surviving bacteria were 10 times more likely to inhibit phytopathogens than bacteria that do not need to inhibit PP to survive.

As outlined in Table 2, both the production of inhibitory compounds and sensitivity to such compounds were unevenly distributed among functional categories. The highest level of inhibitory activity was observed within PP isolates, with 83% able to inhibit one or more isolates, while PC isolates exhibited the lowest level of inhibitory activity (25%).

Among the functional categories analyzed in this study, phytopathogens were by far the most sensitive. PP isolates showed the highest level of sensitivity to inhibitory compounds, with 98% of PP isolates inhibited by one or more other isolates. Fifty two percent of PGPR isolates and 27% of PC strains exhibited sensitivity to the presence of one or more isolates. Phytopathogens were highly targeted by producers in the PGPR category and were the primary target of PC and other PP isolates (Table 2).

TABLE 2
Inhibitory activity and sensitivity observed among cocultured isolates

| | | | Inhibitory | Number | Number | Number |
|---|---|---|---|---|---|---|
| | | Number | Activity | PP | PGPR | PC |
| Category | Genera | Isolates | Detected | Inhibited | Inhibited | Inhibited |
| Phyto- | Erwinia | 20 | 14 | 6 | 8 | 0 |
| pathogens | Pseudomonas | 14 | 13 | 7 | 7 | 3 |
| | Ralstonia | 2 | 1 | 0 | 2 | 0 |
| | Xanthomonas | 16 | 15 | 18 | 7 | 2 |
| | PP Total | 52 | 43 | 26 | 13 | 3 |
| Plant | Alcaligenes | 1 | 1 | 36 | 12 | 1 |
| Growth- | Bacillus | 18 | 12 | 25 | 10 | 1 |
| Promoting | Enterobacter | 29 | 17 | 43 | 32 | 7 |
| Rhizobacteria | Klebsiella | 21 | 0 | 0 | 0 | 0 |
| | Pseudomonas | 12 | 12 | 23 | 17 | 2 |
| | Serratia | 15 | 11 | 46 | 24 | 3 |
| | Acinetobacter | 13 | 11 | 34 | 22 | 5 |
| | PGPR Total | 109 | 64 | 51 | 51 | 12 |
| Plant | Citrobacter | 16 | 7 | 7 | 7 | 6 |
| commensals | Escherichia | 3 | 3 | 3 | 2 | 2 |
| | Hafnia | 10 | 0 | 0 | 0 | 0 |
| | Morganella | 2 | 0 | 0 | 0 | 0 |
| | Pantoea | 1 | 0 | 0 | 0 | 0 |
| | Proteus | 1 | 1 | 7 | 4 | 0 |
| | Providencia | 1 | 0 | 0 | 0 | 0 |
| | Salmonella | 1 | 1 | 7 | 4 | 0 |
| | Stenotrophomonas | 21 | 2 | 20 | 11 | 0 |
| | PC Total | 56 | 14 | 29 | 18 | 7 |
| | Grand Total | 217 | 121 | 51 | 57 | 15 |

In this sample of 217 isolates, it was found that 56% percent of the 217 bacterial isolates exhibited inhibitory activity against one or more isolates. This finding that over half produced a substance that was inhibitory to at least one other isolate is a level of activity that is striking, yet far from the frequent claim by previous studies that 99% of bacteria may produce inhibition due to the presence of a bacteriocin and/or bacteriocins (Riley and Wertz 2002). This discrepancy may be due to the phylogenetic breadth of isolates screened, as most studies of bacteriocins focus on a relatively small number of closely related taxa. It is possible that prior screens have resulted in an inflated frequency of inhibition. These findings could also be a result of the narrow range of bacteriocins; introducing a larger diversity of isolates to the sample may reveal a greater frequency of production.

It was also found that over half of the isolates tested were sensitive to at least one inhibitory substance. Fifty seven percent of the isolates showed inhibition by one or more of the inhibiting isolates. The phytopathogen category was most sensitive, with an astounding 98% of isolates inhibited, which lends support to the theory that there are plenty of antimicrobials produced by bacteria that are capable of serving as an alternative to antibiotics.

### Inhibitory Activity of Plant Pathogens

The plant pathogens showed the highest levels of inhibitory activity, with 83% able to inhibit one or more other isolates. These inhibitory PP were most active against other PP isolates, with 50% of the PP isolates inhibited by one or more PP isolates. The inhibitory PP isolates were far less active against PGPR (12%) or PC (5%) isolates.

Each of the 4 genera including the PP representatives showed high levels of inhibitory activity. Specifically, 93% of Pseudomonas, 94% of Xanthomonas, 70% of Erwinia, and 50% of Ralstonia isolates were inhibitory in this survey (Table 2).

The most inhibitory PP isolate was Xanthomonas vasicola pv. Vasculorum (OS76) which inhibited 15 isolates, 14 of which were other PP isolates. The Vasculorum pathovar was most inhibitory against Ralstonia solancerum, both representative strains showed inhibition. In addition, 25% of Pseudomonas syringae pv tomato and Xanthomonas isolates, as well as 20% of the Erwinia amylovora isolates were inhibited by the Vasculorum pathovar.

Overall, the genera with the highest sensitivity to PP producers were Xanthomonas isolates (75%) and Bacillus (61%) exhibiting sensitivity to one or more inhibitory PP isolates.

### Inhibitory Activity of Plant Growth-Promoting Rhizobacteria

Bacterial strains identified as PGPR exhibited substantial inhibitory activity, with 59% active against one or more of the other isolates. These inhibitory isolates were most active against PP isolates, with 98% of the PP isolates inhibited by one or more PGPR isolates. The inhibitory PGPR strains were less active against other PGPR (47%) or PC (21%) isolates.

The most active PGPR isolate was Enterobacter aerogenes (OS97) which showed 77 instances of inhibition, and was most inhibitory against the plant pathogen Ralstonia solancerum and pathogenic members of the Xanthomonas genus, in which 100% of the isolates were inhibited in both cases. This same isolate also inhibited 89% of PGPR Bacillus, 79% of PP Pseudomonas, 65% of PP Erwinia amylovora, and 41% of PGPR Enterobacter. In fact, this single strain inhibited 81% of all phytopathogen isolates included in the study, while only inhibiting 27% of the PGPR isolates.

The PGPR genera with the highest levels of inhibition were Alcaligenes and Pseudomonas in which 100% of the isolates were inhibitory. Slightly lower levels of inhibition were detected in isolates of Acinetobacter (85%), Serratia (73%), Bacillus (67%), and Enterobacter (56%). Within the inhibitory isolates within these genera, those that showed the highest levels of growth inhibition against PP were Serratia, which inhibited 88% of PP isolates, Enterobacter (83%), and Alcaligenes (69%).

Isolates from the plant pathogen representative sample were highly sensitive to PGPR inhibitory activity, with 98% of the isolates showing sensitivity. All of the Erwinia, Ralstonia, and Xanthomonas isolates were sensitive while 93% of the Pseudomonas isolates were sensitive. For the PC isolates, 100% of the Escherichia and Salmonella were sensitive to PGPR inhibition, while 19% of Stenotrophomonas and Citrobacter, and 10% of Hafnia isolates were sensitive. Finally, 52% percent of PGPR isolates were found to be sensitive to inhibition by other PGPR isolates.

### Inhibitory Activity of Plant Commensals

Bacterial isolates identified as PC were the least active of the isolates surveyed here, with a mere 25% showing inhibitory activity against one or more isolates. Inhibitory PC isolates were most active against PP isolates (56%) and far less active against either PGPR (17%) or PC (13%) isolates.

The PC isolate with the highest inhibitory activity was Stenotrophomonas maltophilia (OS160) which showed 30 instances of inhibition. This isolate was most active against Erwinia amylovora (70%), Bacillus (56%), Ralstonia solanacearum (50%) and P. syringae pv tomato (33%). The genera Escherichia and Citrobacter also showed high levels of inhibition with 100% and 44% inhibitory isolates, respectively. Stenotrophomonas was less likely to be inhibitory (29%). Several PC genera showed no signs of inhibition, specifically Hafnia, Morganella, Pantoea, and Providencia.

PP and PGPR showed 56% and 17% isolated sensitivity, respectively, against PC. The PP genera with the highest sensitivity to PC were Erwinia (70%), Pseudomonas (57%), Ralstonia (50%), and Xanthomonas (38%). The PGPR genera with the highest sensitivity to PC were Bacillus (67%), Klebsiella (14%), Pseudomonas (8%), and Enterobacter (7%).

### Example 2: Phylogenetic Representation of Inhibitory Interactions

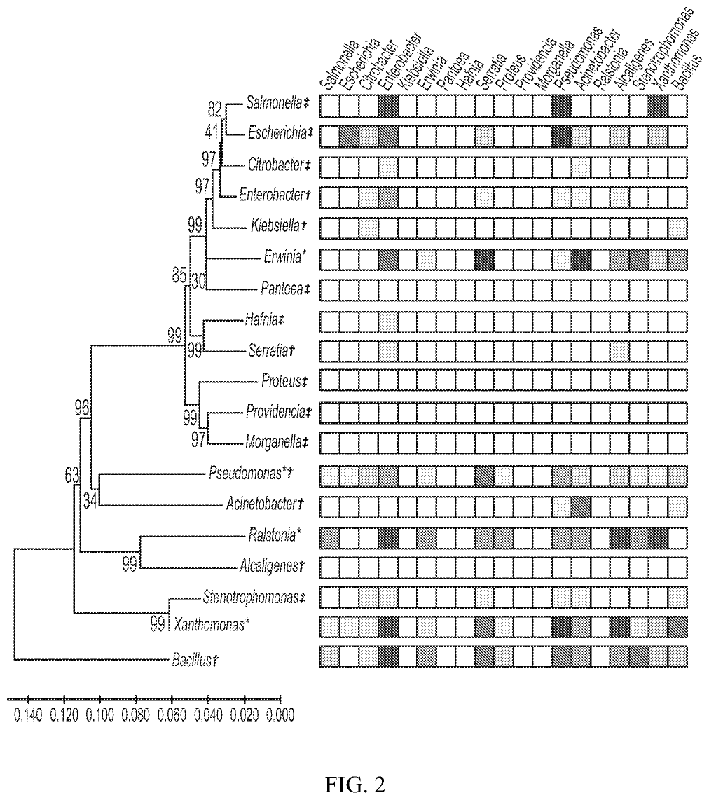

A phylogenetic tree was inferred for the genera included in this study (FIG. 2). The genera containing plant pathogens are identified in bold and the PGPR genera are bold and italicized. A density plot of inhibitory activity was then superimposed onto this phylogeny. The genera along the top of the figure (X-axis) are the potentially inhibitory taxa. Their ability to inhibit members of each genus is plotted against the tips of the phylogenetic tree (Y-axis). The degree of inhibition is indicated in the key and ranges from no inhibition (clear box) to 100% inhibition (darkest grey). For example, Enterobacter produces inhibitory activity capable of inhibiting all Salmonella isolates, while Salmonella is not able to inhibit any of the Enterobacter isolates using the methods described herein.

Ribosomal RNA-based alignments for the species included in this study were obtained from the SILVA high quality ribosomal database. MEGA X was employed to infer a molecular phylogeny using maximum likelihood methods. Confidence in branching patterns was assessed using the bootstrap test with 500 replications.

Several trends emerged in the phylogenetic analysis shown in FIG. 2. An intriguing result was the apparent lack of phylogenetic congruence between the inhibitory taxa and the taxa they inhibit. If, as the bacteriocin literature repeatedly states, isolates are more likely to inhibit their closest relatives (Riley et al. 2003), it would be predicted that the strongest shading would occur along the diagonal of this matrix; inhibitory taxa along the X-axis would more frequently inhibit their closest relatives along the Y-axis. Not only was there an apparent lack of an inhibitory trend along the diagonal in FIG. 2, but the genera displaying the greatest sensitivity to inhibition were often sensitive to their more distant relatives. Instead, we observed that the vast majority of inhibition came from PGPR that were tested on PP isolates (e.g., mutualists vs. pathogens), indicated by the bold outline in FIG. 1. When compared against close-relatives, our method of screening was 3.3 times more likely to produce inhibition, and 4.4 times more likely when compared against intra-niche inhibition among PP isolates.

One example is illustrated within the genus Salmonella. First, focusing on the Y-axis, Salmonella did not inhibit members of its own genus or closely related genera, but did inhibit several of its more distant relatives, such as Pseudomonas, Ralstonia, and Xanthomonas. Second, focusing on the X-axis, Salmonella was not inhibited by most of its closest relatives, such as Escherichia or Citrobacter, but was inhibited by several of its most distant relatives, such as Pseudomonas and Xanthomonas.

The lack of uniform shading along the diagonal in FIG. 2 reveals a discrepancy between the findings regarding the phylogenetic breadth of inhibitory activity and that reported in the bacteriocin literature (Riley et al. 2003). However, there are several cases in the bacterial sample in which a genus is represented by a single isolate, which might skew the phylogenetic visualization.

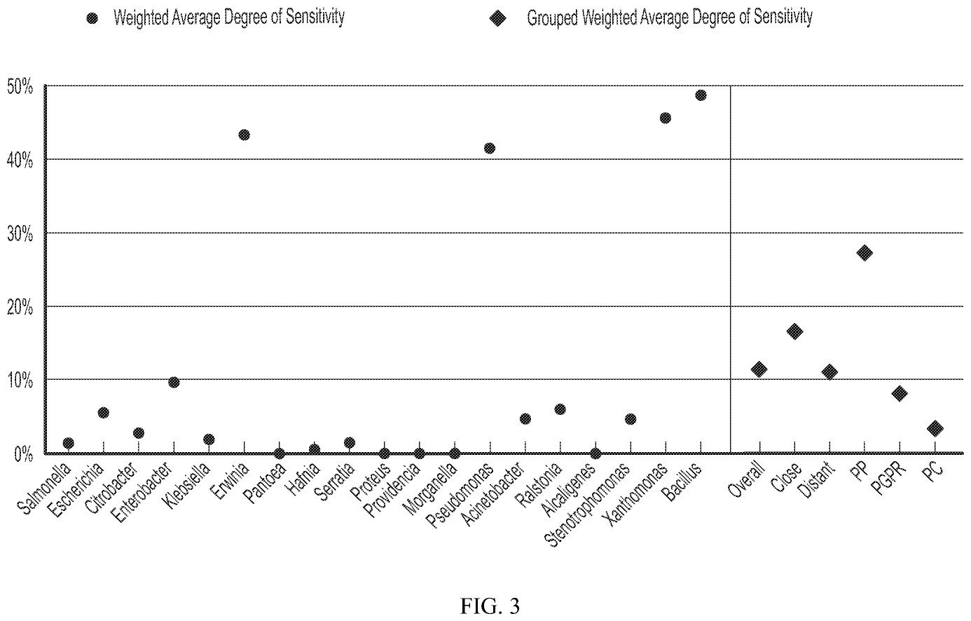

To address this sampling bias, a weighted average degree of sensitivity was calculated by taking the average percent sensitivity across all genera and then weighing the average by sample size. FIG. 3 provides a plot of these values represented individually by each genera, combined as functional groups, and combined as close or distant relatives. The overall weighted average degree of sensitivity was 11%, meaning that every isolate had an 11% chance of being inhibited by any other isolate. The probability of inhibition for close relatives was 16% compared to 11% for distant relatives, and interestingly, the probability for phytopathogenic sensitivity was 26% compared to 9% and 3% for PGPR and PC sensitivity, respectively. For phytopathogens inhibited by PGPR, the weighted average degree of sensitivity was 51%. This shows that there may be a slight trend towards phylogenetic congruence, but it may be dwarfed relative to the enhanced inhibition of PP isolates overall.

According to the phylogenetic analysis shown in FIG. 3, it was found that the inhibitory activity of producers was only slightly correlated with phylogenetic relationship and that plant-associated producers of bacteriocins have a high probability of inhibiting phytopathogens. A potential explanation for this phenomenon is that plant-associated bacteria, which are often mutualistic or symbiotic with their host, derive a selective advantage by inhibiting phytopathogens that would otherwise destroy their host.

The bacteriocin literature is replete with statements to the effect that most bacterial species produce bacteriocins or some other inhibitory agent (Klaenhammer 1988). The inhibition observed in this screen supports that general statement, with all but 5 genera capable of inhibiting isolates in at least one other genera. The most prolific producer of inhibitory agents is Enterobacter, which was inhibitory against members of 10 genera, spanning the phylogenetic diversity included in this sample of isolates (FIG. 3).

### Example 3: Relative Inhibition of PP to PGPR Strains

One of the most significant patterns to emerge in this screen of inhibitory activity is that isolates in certain functional categories (PP, PC, and PGPR) were often preferentially inhibited by isolates in other categories. From an agriculture management perspective, an ideal inhibition pattern would be for an isolate to preferentially inhibit PP isolates, and have no impact or little impact on PGPR isolates. This would permit the inhibition of plant pathogens, while leaving beneficial bacteria relatively undisturbed. To assess this pattern of preferential inhibition, a ratio of inhibitory activity was generated that compares the relative number of PP or PGPR isolates inhibited by members of a species. A ratio of 1 indicates a species shows no preferential inhibition of either PP or PGPR isolates. Species with a ratio greater than I would be considered ideal for agricultural disease management, as this indicates that inhibition of phytopathogens can be accomplished with minimal collateral inhibition of PGPR. Species with a ratio less than 1 would be considered less than ideal for agricultural purposes as the ratio indicates higher levels of inhibition of PGPR isolates than phytopathogens.

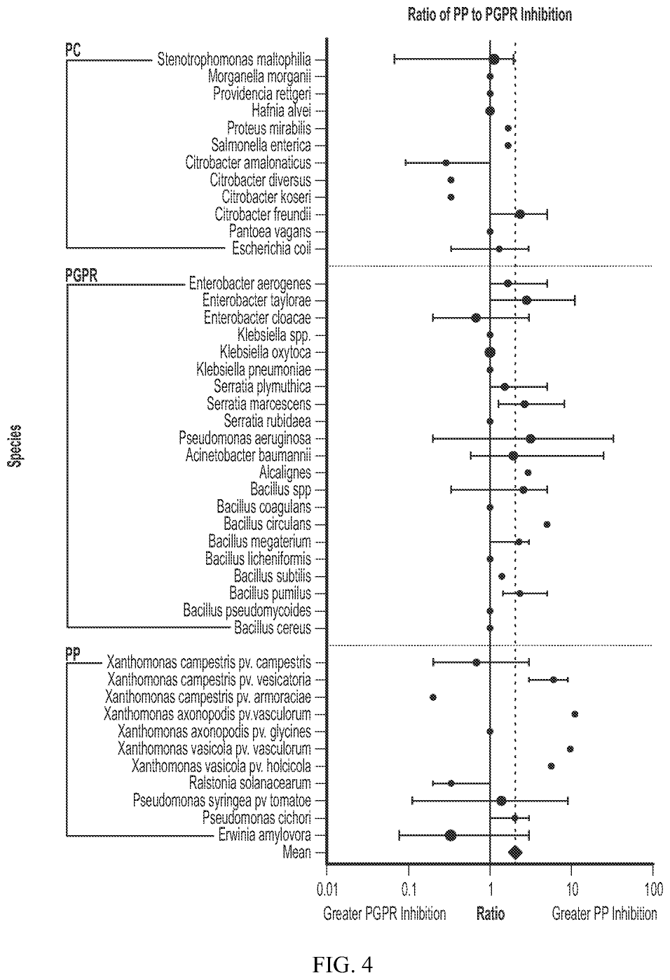

FIG. 4 provides a plot of the ratios of inhibition for the 44 species included in this survey. The ratio for each species is indicated to the right of the species name, with a black dot indicating the average ratio for that species, and the horizontal lines indicating the breadth of ratios calculated for isolates within the species. The average ratio for all plant-associated bacteria is 2.04 (indicated by the vertical line of dots), which reflects preferential inhibition of PP species. PGPR species have an average ratio of 3.04, indicating a strong preferential inhibition of PP species (e.g., PGPR species inhibited approximately 3 PP for every PGPR). PC species have an average ratio of 1.21, demonstrating minimal preferential inhibition of PP species (e.g., PC species inhibited approximately 1.2 PP for every PGPR). Interestingly, PP species, with an average ratio of 1.98, also showed a preferential inhibition of other PP species (e.g., PP species inhibited approximately 2 PP for every PGPR). In light of the previously discussed evolutionary dynamics of plant-associated bacteria (e.g., see Example 1), it makes sense that the ratio of inhibition is greater than 1 across all three species. This is because PGPR bacteria are bacteria that increase the health of their host plant (e.g., make it less likely to die), such that bacteria that live on a plant would be at an evolutionary disadvantage if they produced an antimicrobial substance that killed PGPR.

It is important to note that the range of the ratio of inhibitory activity for each species can be quite large. Some species with an average ratio indicating preferential inhibition of PP also contained individual isolates that preferentially inhibit PGPR, as in the case of Pseudomonas syringae and Pseudomonas aeruginosa. Likewise, species with an average ratio that indicates preferential inhibition of PGPR also contained individual isolates with a strong preference of inhibiting PP, as seen in Erwinia amylovora and Enterobacter cloacae. However, FIG. 4 shows a clear trend towards preferential inhibition of phytopathogens.

Despite inclusion of a wide variety of phytopathogens, with differing phylogenetic location and unrelated target organisms, the isolates in the study were able to inhibit 98% of all phytopathogenic isolates (FIG. 2). Surprisingly, PGPR isolates alone were capable of inhibiting 98% of all phytopathogens tested, and that PC isolates, which were selective for their presumed neutrality, inhibited 56% of the phytopathogens tested (FIG. 2). This level of activity illustrates the abundance of potential antibiotic alternatives, and highlights the effectiveness of the phenotypic screening method. With such high levels of inhibition from PGPR producers, it may presumed they are the most likely category to produce an inhibitory substance, after all, antibiosis is one of the mechanisms that can be used to classify bacteria as PGPR. However, it was found that PGPR isolates were not the most likely to produce an inhibitory substance, but that they were the most effective. Surprisingly, PP isolates were the most likely to produce an inhibitory substance with all but nine isolates (83%) shown to be inhibitory, yet they displayed low efficacy; phytopathogens were only capable of inhibiting 19% of all isolates included in the study, compared to PGPR inhibiting 53%. This is due to the fact that PP producers often inhibited the same isolates, resulting in a high frequency of production but a narrow breadth of inhibition. On average, PP producers were able to inhibit only one isolate each, whereas PGPR producers could inhibit almost two isolates each despite only half of PGPR isolates being producers (Table 2). This result was perplexing, there is no clear selective pressure for phytopathogens that would drive them to frequently produce a relatively ineffective inhibitory substance. This result could be attributed to the PP producers are displaying bacteriocin-like activity as the most inhibited category by PP were other PP isolates, however, the phytopathogens in this study are distantly related. Alternatively, this result could be attributed to competition for similar resources, although the phytopathogens selected affect different target organisms. Nonetheless, the discernible sensitivity of phytopathogen isolates indicated a strong selective pressure for plant-associated bacteria that rely on a host, and provide a framework for the screening of more sustainable antibiotic alternatives.

While isolates in the PGPR category were not the most likely to produce an inhibitory substance, with only 59% of isolates being producers, they did have the greatest inhibitory activity by far (FIG. 2). PGPR producers inhibited 53% of all isolates included in the study and 98% of all PP isolates. However, PGPR isolates were far less likely to produce an inhibitory substance than PP isolates. This result may be due to the fact that a species need not display antibiosis to be classified as a PGPR; biofertilization and biostimulation are other plant growth-promoting mechanisms that can classify a species as PGPR, and may account for the comparatively lower frequency of PGPR producers. For instance, 21 isolates of Klebsiella were included in the study and not a single producing isolate was identified, whereas all 12 isolates of Pseudomonas aeruginosa were identified as producers (Table 2). Consistent with the data, Pseudomonads are known for their ability to produce antimicrobial compounds, and Klebsiella are known to be classified as a PGPR due to nitrogen fixation. Relatively high production of inhibitory substances may have been expected for PGPR, but isolates in the category also displayed an unusual level of sensitivity. Fifty-two percent of isolates in the PGPR category were found to be sensitive which is low in comparison to that of the PP category, but substantial when taking into account the influence of selective pressures (FIG. 2). Since PGPR provide a benefit to the host of the rhizosphere, it should be expected that limited sensitivity as a reduction in PGPR population would indirectly lower the fitness of other rhizosphere inhabitants, due to a direct reduction in fitness of the host. This discrepancy may be resolved by bacteriocin-like activity related to competition over an occupied niche, as the majority of PGPR sensitivity comes from other inhibitory PGPR isolates; PP and PC producers only inhibited 12% and 17% of PGPR isolates, respectively, compared to 47% inhibited by other PGPR isolates. The efficacy and abundance of PGPR producers indicates they are the core reservoir of potential antibiotic alternatives for use in agriculture.

Plant commensals were selected for this study to establish a benchmark of inhibitory activity as they are typically known to be neutral inhabitants of the rhizosphere. PC isolates were the least likely to produce, only 25% of isolates exhibited inhibitory activity compared to 59% of PGPR and 83% of PP. They were also the least sensitive, only 27% of PC isolates were inhibited compared to 52% of PGPR and 98% of PP (Table 2). This comparison highlights that there are few selective pressures to inhibit PC, most likely due to their neutrality, and how relatively sparse their inhibitory activity is. Each functional category is most likely to inhibit PP isolates, and least likely to inhibit PC isolates (Table 2). Interestingly, despite being considered neutral, PC producers inhibited 56% of all phytopathogen isolates which is a greater percent than that inhibited by PP producers themselves (50%) (Table 2). This data adds to the theory that bacterial inhabitants of the rhizosphere face selective pressures to protect their host from threats. Overall, by weighing the results for PP and PGPR against PC, it could be concluded that PGPR isolates are the greatest source of potential antibiotic alternatives, followed by PC and PP.

A phylogenetic analysis of inhibitory activity was conducted to uncover potential trends in the way plant-associated bacteria interact with each other, with an expectation to see a clear visual trend of inhibition among closely related species that would indicate bacteriocin-like activity. Instead it was found that there was no observable trend for closely related species, but that by calculating the weighted average degree of sensitivity, it could be seen that a slight preference for close relatives was observed (16%), as compared to distant relatives (11%) (FIG. 3). A faint visual trend towards inhibition of phytopathogenic genera was, however, observed, as indicated by inconsistent horizontal shading and a calculated probability of inhibition of 26%. These results indicate that there may be slight bacteriocin-like activity (FIG. 3). Alternatively, the theory that plant-associated bacteria may face selective pressure to inhibit pathogenic threats to their host is further supported by this analysis. Phytopathogens were almost twice as likely to be inhibited compared to close relatives, and 2.5 times more likely to be inhibited overall. Phytopathogens were 3 times more likely to be inhibited than PGPR, and 9 times more likely to be inhibited than PC (FIG. 3). When screening phytopathogens against PGPR isolates, phytopathogen isolates were almost 5 times more likely to be inhibited than any isolate in the study, on average. Together, this data demonstrated the methods disclosed herein for use to screen potential antibiotic alternatives with respect to agriculture.

To further explore the agricultural relevance of inhibitory substances, the relative inhibitory profile of each isolate was examined. It was found that, on average, plant-associated bacteria preferentially inhibited phytopathogenic isolates over PGPR isolates in a 2:1 ratio, and that PGPR species in particular displayed a 3:1 ratio of inhibition (FIG. 4). Furthermore, there were individual isolates identified that demonstrated a substantially desirable inhibitory profile; one isolate of Enterobacter aerogenes inhibited 81% of all phytopathogen isolates and only 27% of all PGPR isolates in a 3:1 ratio, and an isolate of Acinetobacter baumannii inhibited 90% of the phytopathogen Erwinia amylovora and only 15% of all PGPR isolates (FIG. 4). These results showed that there are inhibitory substances that could potentially be used to combat phytopathogens with minimal inhibition of beneficial PGPR and that it may be possible to screen for isolates with these desirable characteristics. Antibiotics are losing their efficacy due to resistance, and their breadth of activity suppresses many of the natural benefits provided by PGPR such as increases in plant yield, health, and nutrient content. By identifying inhibitory substances with a narrow or more specific breadth of activity, it may be possible to treat plant disease in the same manner as antibiotics, but with a greater emphasis on sustainability and productivity.

The methods disclosed herein provide a framework for systematically identifying producers of inhibitory substances that display a desirable inhibitory profile, some of which may be bacteriocin-like, by screening plant-associated bacteria based on phylogenetic and evolutionary characteristics. It is clear that there is an abundance of antimicrobials yet to be discovered.

The combination of the findings demonstrate that there is an unexplored opportunity to uncover countless antimicrobials according to the methods disclosed herein, and that some of these antimicrobials may be more be more sustainable or more well-equipped than current treatment methods.

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

What is claimed is:

1. A method of selecting one or more organism-associated bacterial isolates for promoting organism health, comprising:
a. co-culturing each of the bacterial isolates with an organism-associated pathogen;
b. determining the level of inhibition activity for each bacterial isolate against the organism-associated pathogen;
c. co-culturing each of the bacterial isolates with an organism health-promoting bacteria;
d. determining the level of inhibition activity for each bacterial isolate against the organism health-promoting bacteria; and
e. selecting the one or more bacterial isolates for promoting organism health if: the bacterial isolate has inhibition activity against the organism-associated pathogen according to step (b); and the bacterial isolate has substantially no inhibition activity against the organism health-promoting bacteria according to step (d).

2. The method of claim 1, wherein the organism is a non-microbial eukaryote.

3. The method of claim 2, wherein the non-microbial eukaryote is an aquatic organism, a terrestrial organism, a human, or a plant.

4. The method of claim 3, wherein the organism is a plant.

5. The method of claim 4, wherein the organism-associated pathogen is a phytopathogen.

6. The method of claim 4, wherein health-promoting comprises growth-promoting and/or producing a bacteriocin or antimicrobial.

7. The method of claim 4, wherein the organism health-promoting bacteria is a rhizobacteria, a probiotic, or a plant commensal.

8. The method of claim 1, wherein substantially no inhibition comprises a zone of inhibition of less than 5 mm.

9. The method of claim 7, wherein the method comprises:
selecting one or more plant-associated bacterial isolates for promoting plant health, comprising:
a. co-culturing each of the bacterial isolates with a phytopathogen;
b. determining the level of inhibition activity for each bacterial isolate against the phytopathogen;
c. co-culturing each of the bacterial isolates with a plant growth-promoting rhizobacteria, a probiotic, or a plant commensal;
d. determining the level of inhibition activity for each bacterial isolate against the plant growth-promoting rhizobacteria, probiotic, or plant commensal; and
e. selecting the one or more bacterial isolates for promoting plant health if: the bacterial isolate has inhibition activity against the phytopathogen according to step (b); and the bacterial isolate has substantially no inhibition activity against the plant growth-promoting rhizobacteria, probiotic, or plant commensal according to step (d).

10. The method of claim 9, wherein promoting plant health comprises promoting one or more plant health parameters selected from the following list:
(i) disease resistance;
(ii) the ability to produce or change the concentration of indoleacetic acid, gibberellic acid, cytokinins and/or ethylene;
(iii) the ability to perform asymbiotic N2 fixation;
(iv) the ability to produce siderophores, antibiotics, or cyanide;
(v) the ability to solubilize mineral phosphates and other nutrients; and
(vi) the ability to change performance of symbiotic N2 fixation, nodulation, or nodule occupancy.

11. The method of claim 9, wherein the phytopathogen comprises Erwinia amylovora, Agrobacterium tumefaciens, Pseudomonas syringae pv. tomato or other P. syringae pathovars, P. cichorii, Xanthomonas campestris pv. armoraciae and pv. vesicatoria, Ralstonia solanacearum, X. axonopodis pv. glycines, X. axonopodis pv. vasculorum, X. vasicola pv. holcicola, X. vasicola pv. vasculorum, X. campestris pv. campestris or other pathogenic Xanthomonas, or Xylella fastidiosa.

12. The method of claim 9, wherein the plant growth-promoting rhizobacteria comprises Alcaligens species, Arthrobacter species, Bacillus cereus, B. circulans, B. coagulans, B. licheniformis, B. megaterium, B. pseudomycoides, B. pumilus, and other Bacillus species, Enterobacter aerogenes, E. cloacae, E. taylorae, Klebsiella oxytoca, K. pneumoniae, P. aeruginosa and other Pseudomonas species, Paraburkholderia species, Serratia marcescens, S. plymuthica, S. rubidaea and other Serratia species, and Acinetobacter baumannii or other Acinetobacter species.

13. The method of claim 9, wherein the plant commensal comprises Arabidopsis species, Citrobacter amalonaticus, C. freundii, C. koseri or other Citrobacter species, Escherichia coli, Hafnia alvei or other Hafnia species, Morganella morganii or other Morganella species, Pantoea vagans or other Pantoea species, Proteus mirabilis, Providencia rettgeri, Salmonella enterica or other Salmonella species, or Stenotrophomonas maltophili.

14. The method of claim 9, wherein co-culturing comprises co-culturing on a transwell plate. in a microfluidic platform, on a solid support, or in a broth.

15. The method of claim 14, wherein co-culturing comprises co-culturing on a solid support.

16. The method of claim 15, wherein a solid support comprises a plate, a three-dimensional scaffold, a hydrogel, or a microarray.

17. The method of claim 16, wherein the plate is a nutrient agar plate.

18. The method of claim 14, wherein co-culturing comprises co-culturing in a broth.

19. A method of selecting one or more plant-associated bacterial isolates for promoting plant health, comprising:
a. co-culturing each of the bacterial isolates with at least 5 different species of phytopathogens;
b. determining the level of inhibition activity for each bacterial isolate against each phytopathogen;
c. co-culturing each of the bacterial isolates with at least 5 different species of plant growth-promoting rhizobacteria, probiotics, and/or plant commensals;
d. determining the level of inhibition activity for each bacterial isolate against each plant growth-promoting rhizobacteria, probiotic, or plant commensal; and
e. selecting the one or more bacterial isolates for promoting plant health if: the one or more bacterial isolates has inhibition activity against each of the phytopathogens according to step (b); and the one or more bacterial isolates has substantially no inhibition activity against each of the plant growth-promoting rhizobacteria, probiotic, or plant commensal according to step (d).

20. The method of claim 19, further comprising determining a ratio of inhibitory activity for each of the one or more bacterial isolates and selecting the one or more bacterial isolates for promoting plant health if the ratio is greater than 1.

21. The method of claim 19, wherein substantially no inhibition comprises a zone of inhibition of less than 5 mm.

22. A method of determining whether one or more plant-associated bacterial isolates is likely to have inhibition activity against a phytopathogen, comprising:
a. obtaining a control bacterial isolate having inhibition activity against the phytopathogen;
b. determining the phylogenetic distance for each of the one or more plant-associated bacterial isolates against the control bacterial isolate; and
c. determining the one or more plant-associated bacterial isolates as likely to have inhibition activity against the phytopathogen if the phylogenetic distance to the control bacterial isolate according to step (b) is less than or equal to 0.040.
